(19) United States
(12) Patent Application Publication
(10) Pub. No.: US 2003/0215947 A1
(43) Pub. Date: Nov. 20, 2003

(54) Methods for enhancing targeted gene alteration using oligonucleotides

(76) Inventors: Eric B. Kmiec, Landenberg, PA (US); Howard B. Gamper, Philadelphia, PA (US); Michael C. Rice, Newtown, PA (US); Li Liu, Newark, DE (US)

Correspondence Address: FISH & NEAVE, 1251 AVENUE OF THE AMERICAS, 50TH FLOOR, NEW YORK, NY, 10020-1105, US

(21) Appl. No.: 10/351,662

(22) Filed: Jan. 24, 2003

Related U.S. Application Data
(63) Continuation of application No. PCT/US01/23770, filed on Jul. 27, 2001.
(60) Provisional application No. 60/220,999, filed on Jul. 27, 2000.
(60) Provisional application No. 60/244,989, filed on Oct. 30, 2000.

(51) Int. Cl. C12N 15/85; C12N 15/82; C12N 15/74

(52) U.S. Cl. 435/455; 435/468; 435/483

(57) ABSTRACT

Methods are presented for enhancing the efficiency of oligonucleotide-medidated repair or alteration of genetic information. The methods comprise using cells or cell-free extracts having altered levels or activity of at least one protein from the RAD52 epistasis group, the mismatch repair group or the nucleotide excision repair group. Kits and compositions are also presented.

### FIELD OF THE INVENTION

This invention relates to oligonucleotide-directed repair or alteration of genetic information and methods and compositions for enhancing the efficiency of such alteration.

### BACKGROUND OF THE INVENTION

A number of different poly- and oligo-nucleotides have been described for use in the alteration of genomic DNA including chimeric RNA-DNA oligonucleotides that fold into a double-stranded, double hairpin conformation and single-stranded chemically modified oligonucleotides. For examples of chimeric RNA-DNA double-stranded hairpin oligonucleotides useful in the methods of the invention, see, for example, U.S. Pat. No. 5,945,339, “Methods to Promote Homologous Recombination in Eukaryotic Cells and Organisms”; U.S. Pat. No. 5,795,972, “Chimeric Mutational Vectors Having Non-Natural Nucleotides”; U.S. Pat. No. 5,871,984, “Compounds and Methods for Site Directed Mutations in Eukaryotic Cells”, and U.S. patent application Ser. No. 60/220,999, “Methods for Enhancing Gene Conversion or Genetic Repair by Chimeric Oligonucleotides,” filed Jul. 27, 2000, which are all incorporated by reference herein in their entirety. For examples of single-stranded chemically modified oligonucleotides useful in the methods of the invention, see U.S. patent application Ser. No. 60/244,989, “Targeted Chromosomal Genomic Alterations with Modified Single Stranded Oligonucleotides,” filed Oct. 30, 2000, International Patent Application PCT/US01/09761 “Targeted Chromosomal Genomic Alterations with Modified Single Stranded Oligonucleotides,” filed Mar. 27, 2001, and International Patent Application PCT/US01/17672 “Targeted Chromosomal Genomic Alterations in Plants Using Modified Single-Stranded Oligonucleotides,” filed Jun. 1, 2001, which are all incorporated by reference herein in their entirety. These oligonucleotides have been shown to effect targeted alteration of single base pairs as well as frameshift alterations in a variety of host organisms, including bacteria, fungi, plants and animals.

Without being limited by theory, it is believed that DNA repair pathways are involved in oligonucleotide-directed gene alteration. Several cellular pathways and gene groups are believed to be involved in mediating in vivo repair of DNA lesions resulting from radiation or chemical mutagenesis, including the RAD52 epistasis group of proteins, the mismatch repair group of proteins or the nucleotide excision repair group of proteins. The role of these proteins in homologous recombination and maintaining genome integrity has been extensively studied and is reviewed, for example, in Heyer, Experientia 50(3), 223-233 (1994); Thacker, Trends in Genetics 15(5), 166-168 (1999); Paques & Hasber, Microbiol. and Molec. Biol. Rev. 63(2), 349-404 (1999); and Thompson & Schild, Mutation Res. 477, 131-153 (2001). The specific function of these proteins in oligonucleotide-directed gene alteration is not well understood. Moreover, because oligonucleotide-directed gene alteration relies on the activity of molecules that have alternative chemistries as compared to products resulting from radiation or chemical mutagens, whether any of the same proteins would be involved was unknown and unpredictable.

The present invention analyzes proteins and genes in the RAD52 epistasis group, the mismatch repair group or the nucleotide excision repair group. Members of these groups include: RAD50, RAD51, RAD52, RAD54, RAD55, RAD57, RAD59, MRE11 and XRS1 in the RAD52 epistasis group; MSH2, MSH3, MSH6 and PMS1 in the mismatch repair group; and RAD1, RAD2, RAD10, RAD23 and EXO1 in the nucleotide excision repair group. These proteins function through multiple complex interactions.

The utility of oligonucleotide-mediated gene alteration as a means, for example, to generate agricultural products with enhanced traits or to generate animal models or animals with desired traits is diminished by its relatively low frequency. A need exists for methods to enhance the efficiency of oligonucleotide-mediated gene alteration. The present invention concerns such methods and compositions for enhancing both in vivo and in vitro gene alteration using oligonucleotides.

In yet another embodiment, the invention relates to kits comprising a cell or cell-free extract with reduced levels or activity of at least one of the RAD1, RAD51, RAD52, RAD57 or PMS proteins. In another embodiment, the kit further comprises an oligonucleotide capable of directing gene alteration.

### SUMMARY OF THE INVENTION

The invention involves methods of targeted gene alteration comprising administering to a cell or tissue from a fungus, a plant, or an animal an oligonucleotide having a gene alteration sequence wherein the target cell or tissue has altered levels or activity of at least one protein from the RAD52 epistasis group, the mismatch repair group or the nucleotide excision repair group. The invention also involves methods of gene alteration using cell-free extracts having altered levels or activity of at least one protein from the RAD52 epistasis group, the mismatch repair group or the nucleotide excision repair group. Altering the levels or activity of these proteins can be achieved by any means known to one of skill in the art, including, for example, using inhibitors of the activity of one of the proteins, suppressors of expression of one of the genes, a mutation in one of the genes that alters the expression or the activity of the protein, and addition of extra copies of one of the proteins or genes.

In another embodiment, the present invention relates to processes to alter plant genomes by administering to a plant cell or tissue at least one oligonucleotide having a desired gene alteration sequence, wherein the plant cell has altered levels and/or activity of a protein encoded by a plant homolog, ortholog or paralog of a RAD1, RAD51, RAD52, RAD57 or PMS1 gene. Such plant cells can then be used to generate plants which are a further embodiment of the invention. The invention further relates to methods for genetically altering plants to enhance or generate desirable traits, for example herbicide or pest resistance.

In a further embodiment, the present invention relates to a process to genetically alter animals, particularly livestock, to enhance expression of desirable traits, comprising administering to a target cell at least one oligonucleotide having a gene alteration sequence, wherein the cell has altered levels and/or activity of a protein encoded by a gene homologous to the RAD1, RAD51, RAD52, RAD57 or PMS1 gene from yeast and the animals produced thereby.

In a further embodiment, the present invention relates to an assay to identify inhibitors of RAD1, RAD51, RAD52, RAD57 and/or PMS1 protein activity and/or one or more suppressors of a RAD1, RAD51, RAD52, RAD57 or PMS1 gene expression comprising contacting a sample with an oligonucleotide in a system known to provide for gene alteration and measuring whether the amount of gene alteration is less, more, or the same as in the absence of sample.

### DETAILED DESCRIPTION OF THE INVENTION

The present invention involves methods of gene alteration comprising administering to a cell or tissue from a bacterium, a fungus, a plant, or an animal an oligonucleotide having a gene alteration sequence wherein the target cell or tissue has altered levels or activity of at least one protein from the RAD52 epistasis group, the mismatch repair group or the nucleotide excision repair group. Altering the levels or activity of the proteins can be achieved by any means known to one of skill in the art, including, for example, inhibiting the activity of one of the proteins, suppressing expression of one of the genes, introducing a mutation in one of the genes that alters expression or activity of the protein, and adding extra copies of one of the proteins.

The methods of the present invention can be used with any oligonucleotide having gene alteration activity including, for example, chimeric, RNA-DNA double hairpin oligonucleotides and modified, single-stranded oligonucleotides. Such oligonucleotides are described, for example, in U.S. Pat. No. 5,945,339; U.S. Pat. No. 5,795,972; U.S. Pat. No. 5,871,984 and International Patent Application PCT/US01/09761 which are hereby incorporated by reference in their entirety. Oligonucleotides designed to direct gene alteration comprise a portion that is generally identical in sequence to a portion of a gene or a portion of the complement of a gene except for the specific difference designed to direct gene alteration. Thus, the oligonucleotides used in the methods of the invention have at least one base pair different from the sequence of the target gene, or have at least one base pair different from the complement of the DNA sequence of the target gene. The methods of the invention can be used to enhance the alteration mediated by an oligonucleotide directing any kind of alteration, including, for example, deletion, insertion or replacement of 1, 2 or 3 consecutive nucleotides in the target sequence. Further, gene alteration by oligonucleotides targeting 1, 2, or 3 multiple sequence alterations is also enhanced using the methods of the instant invention. Each of such multiple mutations can include, for example, deletion, insertion or replacement of 1, 2 or 3 consecutive nucleotides in the target sequence. Where gene alteration of multiple sequence targets are enhanced, the multiple alterations can be directed by a single oligonucleotide or by 1, 2 or 3 separate oligonucleotides. In a preferred embodiment, the multiple alterations are directed by a single oligonucleotide.

The oligonucleotides can be introduced into cells or tissues by any technique known to one of skill in the art. Such techniques include, for example, electroporation, liposome transfer, naked nucleic acid insertion, particle bombardment and calcium phosphate precipitation. In one embodiment the transfection is performed with a liposomal transfer compound, for example, DOTAP (N-1-(2,3-Dioleoyloxy)propyl-N,N,N-trimethylammonium methylsulfate, Boehringer-Mannheim) or an equivalent, such as LIPOFECTIN®. In another embodiment, the transfection technique uses cationic lipids. The methods of the invention can be used with a wide range of concentration of oligonucleotides. For example, good results can be achieved with 10 nM/10⁵ cells. A ratio of about 500 ng of oligonucleotide in 3 μg of DOTAP per 10⁵ cells can be used. The transfected cells may be cultured in different media, including, for example, in serum-free media, media supplemented with human serum albumin or human serum.

The methods of the instant invention can be used to enhance the efficiency of gene alteration directed by an oligonucleotide that targets either strand of a double-stranded target nucleic acid. The methods of the invention can be used to enhance the efficiency of an oligonucleotide targeting any part of a gene including, for example, an exon, an intron, a promoter and a 3′- or 5′-untranslated region. Further, the methods of the invention can be used to enhance the efficiency of an oligonucleotide targeting intragenic sequences. In a preferred embodiment, these methods are used to enhance the efficiency of an oligonucleotide targeting actively transcribed sequences. In another preferred embodiment, these methods are used to enhance the efficiency of an oligonucleotide targeting the non-transcribed strand of the target sequence.

The methods of the invention involve the alteration of the expression or the activity of at least one protein selected from the group consisting of the RAD52 epistasis group proteins RAD51, RAD52, and RAD57; the mismatch repair group protein PMS1; and the nucleotide excision repair group protein RAD1. The symbols for these proteins are taken from the yeast (Saccharomyces cerevisiae) designations, but it is understood that homologs, orthologs and paralogs from other organisms, including bacteria, plants, animals and other fungi can be used in the methods of the instant invention. Example sequences from bacteria and fungi include mutL from Bacillus subtilis (GenBank™ Acc. No. P49850), rhp57 from Schizosaccharomyces pombe (GenBank™ Acc. No. T43507), uvsC from Emericella nidulans

(GenBank™ Acc. No. CAB02454), pms1 from S. pombe (GenBank™ Acc. No. P54280), MUS38 from Neurospora crassa (GenBank™ Acc. No. BM28847), RAD52 from Kluyveromyces lactis (GenBank™ Acc. No. P41768), RAD52 from S. pombe (GenBank™ Acc. No. P36592), MUS11 from N. crassa (GenBank™ Acc. No. BAB13343), RAD 51 from S. pombe (GenBank™ Acc. No. P36601), RAD51 from Ustilago maydis (GenBank™ Acc. No. Q99133), RAD51 from E. nidulans (GenBank™ Acc. No. P78579) and RAD51 from Penicillium paxilli (GenBank™ Acc. No. BAA92869). Example sequences from plants include DMC1 from Glycine max (GenBank™ Acc. No. Q96449), MUA2.3 from Arabidopsis thaliana (GenBank™ Acc. No. BAB08781), UVH1 from A. thaliana (GenBank™ Acc. No. AAF01274), RAD51 from A. thaliana (GenBank™ Acc. No. P94102) and RAD51 from Lycopersicon esculentum (GenBank™ Acc. No. Q40134). Example sequences from animals, including humans, include human XRCC3 (GenBank™ Acc. No. AAC04805), mouse RAD51 (GenBank™ Acc. No. NP_{—}033040), spindle B from Drosophila melanogaster (GenBank™ Acc. No. AAC42663), human PMS2 (GenBank™ Acc. No. P54278), mouse PMS2 (GenBank™ Acc. No. P54279), human PMS1 (GenBank™ Acc. No. P54277), human MLH1 (GenBank™ Acc. No. P40692), ERCC4 from Cricetulus griseus (GenBank™ Acc. No. BAA89229), human ERCC4 (GenBank™ Acc. No. NP_{—}005227), mouse xpf (GenBank™ Acc. No. NP_{—}056584), mei-9 from D. melanogaster (GenBank™ Acc. No. MF45938), human RAD52 (GenBank™ Acc. No. AAF05533), mouse RAD52 (GenBank™ Acc. No. P43352), chicken RAD52 (GenBank™ Acc. No. P39022), RAD51 from C. griseus (GenBank™ Acc. No. P70099), human RAD51 (GenBank™ Acc. No. Q06609), chicken RAD51 (GenBank™ Acc. No. P37383), mouse RAD51 (GenBank™ Acc. No. 035719), rabbit RAD51 (GenBank™ Acc. No. 077507), RAD51 from Xenopus laevis (GenBank™ Acc. No. Q91918), RAD51 from Bombyx mori (GenBank™ Acc. No.001679) and RAD51 from D. melanogaster (GenBank™ Acc. No. Q27297).

The alteration of the expression or the activity of the at least one protein can be either increasing or reducing the expression or activity of the protein. Where the alteration is increasing the expression or the activity, the increase in expression or activity can be about one, two, three, four, five, six, seven, eight, nine, ten, twelve, fifteen, twenty, thirty, and fifty or more fold. Similarly, where the alteration is reducing the expression or the activity, the decrease in expression or activity can be about one, two, three, four, five, six, seven, eight, nine, ten, twelve, fifteen, twenty, thirty, and fifty or more fold. Reducing the expression or the activity of the protein can also be achieved by completely eliminating the expression or the activity of the target protein.

Any method for reducing the expression or the activity of the above-described proteins known to one of skill in the art can be employed in the methods of the instant invention. Methods for reducing or eliminating expression of the activity of the above-described proteins include generating mutations in the targeted gene from the RAD52 epistasis group gene, mismatch repair group gene or nucleotide excision repair group gene. Such mutations may be engineered in the host organism using any method known to those of skill in the art, including, for example, using a chimeric, RNA-DNA double hairpin or modified, single-stranded oligonucleotide; isolating a spontaneous mutation; and selecting/screening from a mutagenized population. These methods can be combined, for example, to identify a useful mutation in one organism and then engineering the specific mutation in a homolog, ortholog or paralog of a second organism. Any type of mutation in the RAD52 epistasis group gene, mismatch repair group gene or nucleotide excision repair group gene can be used for the methods of the instant invention including, for example, missense, deletion, insertion, transposon, and retroposon.

Methods for reducing or eliminating expression of the activity of the above-described proteins also include engineering extragenic elements, including, for example, antisense methods, ribozyme methods, cosuppression, gene silencing methods, RNA interference (“RNAi”) methods, and using triplex-forming oligonucleotides.

Antisense methods involve the introduction or expression of a nucleic acid molecule that is complementary to a transcript encoding the protein. This nucleic acid molecule does not need to be 100% complementary to the target transcript, but can exhibit a limited degree of complementarity. Preferably the antisense nucleic acid molecule is at least 90% and more preferably at least 95% complementary to the target transcript. In order to cause an antisense-effect, antisense oligonucleotides are 10-40 nucleotides long, preferably from 15-30 nucleotides in length. For in vivo expressed antisense, oligonucleotides preferably have a length of at least 100 nucleotides and more preferably a length of at least 500 nucleotides. It is also preferred that in vivo expressed antisense nucleic acid molecules are less than about 5000 nucleotides in length, more preferably less than about 2500 nucleotides. Such antisense polynucleotides can be produced in vivo by transcription or they can be introduced as oligo- or polynucleotides. For antisense oligonucleotides, it is preferred that the oligonucleotides comprise at least PNA, LNA, or 2′-O-methyl RNA residue or at least one phosphorothioate backbone linkage to reduce their degradation.

The antisense nucleic acid molecules useful as suppressors of gene expression in the methods of the invention are typically administered to a subject or generated in situ such that they hybridize with or bind to cellular mRNA encoding a polypeptide to thereby inhibit expression of the polypeptide, for example, by inhibiting transcription and/or translation. The hybridization is generally by conventional nucleotide complementarity to form a stable duplex. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, for example, by linking the antisense nucleic acid molecule to a peptide or an antibody which binds to a cell surface receptor or antigen. The antisense nucleic acid molecule can also be delivered to cells using one of the methods for delivering the gene altering oligonucleotides as described herein. To achieve sufficient intracellular concentrations of the antisense molecules, vector constructs in which the antisense nucleic acid molecule is placed under the control of a strong promoter are preferred.

Cosuppression methods relate to RNA molecules which reduce the expression in a host cell of the nucleic acid molecule encoding the target protein due to a cosuppression-effect. The principle of the cosuppression as well as the production of corresponding DNA sequences is precisely described, for example, in WO 90/12084. Such DNA molecules preferably encode an RNA having a high degree of homology to the target transcript. While cosuppressing RNA molecules are the same sense as an RNA molecule encoding the protein, it is not necessary for cosuppression that the RNA molecule actually encodes a polypeptide. For example, an RNA with nonsense mutations but substantial sequence similarity to the target nucleic acid molecule can effectively cosuppress.

RNAi refers to the introduction of homologous double-stranded RNA (dsRNA) to specifically target a gene transcript, resulting in null or hypomorphic levels of the resulting protein. In contrast to antisense methods, rather than single-stranded antisense RNA, a double-stranded RNA interferes with expression of the target. Further, RNAi methods are highly sequence-specific and very sensitive with only a few dsRNA molecules required per cell for effective interference.

Yet another method for reducing the expression or the activity of the target protein involves RNA molecules with a ribozyme activity which specifically cleave transcripts encoding one of the above-described DNA repair proteins. Ribozymes are catalytically active RNA molecules capable of cleaving RNA molecules and specific target sequences. There are various classes of ribozymes, but the group I intron type and the “hammerhead” motif type are preferred for the methods of the invention. The specific recognition of the target RNA molecule may be modified by altering the sequences flanking this motif. These recognition sequences pair with sequences in the target molecule and determine the position of cleavage in the target molecule. The sequence requirements for efficient cleavage are extremely low and a specific ribozyme can be designed for almost any desired target.

Alternatively, gene expression can be inhibited by targeting nucleotide sequences complementary to the regulatory region of the target gene (e.g., a promoter and/or enhancer) to form triple helical structures that prevent transcription of the gene in target cells. For example, using a nucleic acid molecule which binds to DNA duplexes through specific interactions with the double helix. Such nucleic acid molecules are generally from 12-40 nucleotides in length and preferably from 25-35 nucleotides in length. See, generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

Surprisingly, different alterations in the levels or activity of the proteins of the RAD52 epistasis group, mismatch repair group or nucleotide excision repair group influence the efficiency of gene alteration differently. As disclosed herein, depending on the type of gene alteration targeted (e.g. deletion, insertion, or replacement), alterations in the levels or activity of particular proteins of the RAD52 epistasis group, mismatch repair group or nucleotide excision repair group are preferred. For example, the instant application demonstrates that it is preferred that the levels or activity of a protein selected from RAD1, RAD51/52, RAD57 and PMS1 is reduced for methods of the invention using oligonucleotides targeting in vivo replacement. Among these, PMS1 is most preferred. Similarly, for methods using oligonucleotides targeting in vivo insertion, it is preferred that the levels or activity of the PMS1 protein are reduced.

The examples in this application further demonstrate methods and assay systems to identify and optimize which background mutations and/or activity reductions to use to achieve enhanced gene alteration efficiency for an oligonucleotide that introduces a desired target gene alteration, including, for example, an insertion, deletion, or replacement alteration as described herein as well as oligonucleotides that introduce multiple gene alterations. One of skill in the art could readily modify one of these systems to assay correction of any target to optimize the strain background for introduction of desired gene alterations using the teachings of this application.

The methods of the instant invention can also be used to enhance the efficiency of gene alteration in vitro using cell-free extracts. The cell-free extract can be derived from cells or tissue from any organism including bacteria, fungi, plants, and animals, including humans or other mammals. Cells or cell-free extracts for use in the methods and compositions of the invention include, for example, cultured cells of human liver, lung, colon, cervix, kidney, epithelium. Additional cells or cell-free extracts for use in the methods and compositions of the invention include, for example, COS-1 and COS-7 cells (African green monkey), CHO-K1 cells (Chinese hamster ovary), H1299 cells (human epithelial carcinoma, non-small cell lung cancer), C1271 (immortal murine mammary epithelial cells), MEF (mouse embryonic fibroblasts), HEC-1-A (human uterine carcinoma), HCT15 (human colon cancer), HCT1 16 (human colon carcinoma), LoVo (human colon adenocarcinoma), and HeLa (human cervical carcinoma) cancer cells as well as PG12 cells (rat pheochromocytoma) and ES cells (human embryonic stem cells). The extract can be derived from any source, including, for example cultured cells, primary isolated cells, or tissue. The extract can be derived from a cell or tissue, wherein the levels or activity in the extract of at least one protein selected from the group consisting of RAD1, RAD51, RAD52, RAD57 or PMS1 is altered.

As with the in vivo methods of the invention, the alteration in cell-free extracts of the expression or the activity of the at least one protein can be either increasing or reducing the expression or activity of the protein. Where the alteration is increasing the expression or the activity, the increase in expression or activity can be about one, two, three, four, five, six, seven, eight, nine, ten, twelve, fifteen, twenty, thirty, and fifty or more fold. Similarly, where the alteration is reducing the expression or the activity, the decrease in expression or activity can be about one, two, three, four, five, six, seven, eight, nine, ten, twelve, fifteen, twenty, thirty, and fifty or more fold. Reducing the expression or the activity of the protein can also be achieved by completely eliminating the expression or the activity of the target protein.

Cell-free extract with reduced levels and/or activity of the protein can be used in the methods of the invention. For this purpose, any method known to one of skill in the art, including the methods described herein, can be employed to decrease the expression or the activity of the proteins in the cell before obtaining the cell-free extract therefrom. Further it is possible to reduce the levels or the activity of the protein by depleting the protein from the cell-free extract by any means known to one of skill in the art. The depletion can be achieved by, for example, addition of extra copies of interacting proteins, immunoprecipitation, immunosequestration, or specific degradation of the target protein.

Methods of enhancing gene alteration using cell-free extracts with altered levels or activity of at least one protein selected from the group consisting of RAD1, RAD51, RAD52, RAD57 or PMS1 are particularly useful for directed alteration of isolated episomal targets, including, for example, plasmids, cosmids, artificial chromosomes, YACs, BACs, PLACs, and BiBACS. However, the in vitro methods may be used with any target nucleic acid molecule. Similarly, methods of the invention for enhancing gene alteration alteration in vivo can be used with any target nucleic acid molecule in cells, including, for example, genomic or chromosomal targets, organellar genomic targets, and episomal targets.

The present invention relates to a process to genetically alter animals, including livestock, to enhance expression of desirable traits, comprising administering to a target cell at least one oligonucleotide having a gene alteration sequence, wherein the cell has altered levels and/or activity of a protein encoded by a gene that is a homolog, ortholog or paralog of a RAD1, RAD51, RAD52, RAD57 or PMS1 gene from yeast and the animals produced thereby. The methods of the invention can be used to genetically alter cells from any animal, including, for example, horses, cattle, sheep, pigs, goats, bison; fowl such as chickens, geese, ducks, turkeys, pheasant, ostrich and pigeon; fish such as salmon, tilapia, catfish, trout and bass; model experimental animals such as mice, rats and rabbits; and pets such as dogs and cats.

The present invention encompasses methods for introducing targeted gene alterations in plants using an effective amount of at least one oligonucleotide containing a gene alteration sequence in a plant strain having reduced levels and/or activity of at least one protein encoded by a plant homolog of a RAD1, RAD51, RAD52, RAD57 or PMS1 gene or suppressors of a RAD1, RAD51, RAD52, RAD57 or PMS1 gene. Preferred target plants include, for example, experimental model plants such as Chlamydomonas reinhardtii, Physcomitrella patens, and Arabidopsis thaliana in addition to crop plants such as cauliflower (Brassica oleracea), artichoke (Cynara scolymus), fruits such as apples (Malus, e.g. domesticus), mangoes (Mangifera, e.g. indica), banana (Musa, e.g. acuminata), berries (such as currant, Ribes, e.g. rubrum), kiwifruit (Actinidia, e.g. chinensis), grapes (Vitis, e.g. vinifera), bell peppers (Capsicum, e.g. annuum), cherries (such as the sweet cherry, Prunus, e.g. avium), cucumber (Cucumis, e.g. sativus), melons (Cucumis, e.g. melo), nuts (such as walnut, Juglans, e.g. regia; peanut, Arachis hypogeae), orange (Citrus, e.g. maxima), peach (Prunus, e.g. persica), pear (Pyra, e.g. communis), plum (Prunus, e.g. domestica), strawberry (Fragaria, e.g. moschata or vesca), tomato (Lycopersicon, e.g. esculentum); leaves and forage, such as alfalfa (Medicago, e.g. sativa or truncatula), cabbage (e.g. Brassica oleracea), endive (Cichoreum, e.g. endivia), leek (Allium, e.g. porrum), lettuce (Lactuca, e.g. sativa), spinach (Spinacia, e.g. oleraceae), tobacco (Nicotiana, e.g. tabacum); roots, such as arrowroot (Maranta, e.g. arundinacea), beet (Beta, e.g. vulgaris), carrot (Daucus, e.g. carota), cassava (Manihot, e.g. esculenta), turnip (Brassica, e.g. rapa), radish (Raphanus, e.g. sativus), yam (Dioscorea, e.g. esculenta), sweet potato (Ipomoea batatas); seeds, including oilseeds, such as beans (Phaseolus, e.g. vulgaris), pea (Pisum, e.g. sativum), soybean (Glycine, e.g. max), cowpea (Vigna unguiculata), mothbean (Vigna aconitifolia), wheat (Triticum, e.g. aestivum), sorghum (Sorghum e.g. bicolor), barley (Hordeum, e.g. vulgare), corn (Zea, e.g. mays), rice (Oryza, e.g. sativa), rapeseed (Brassica napus), millet (Panicum sp.), sunflower (Helianthus annuus), oats (Avena sativa), chickpea (Cicer, e.g. arietinum); tubers, such as kohirabi (Brassica, e.g. oleraceae), potato (Solanum, e.g. tuberosum) and the like; fiber and wood plants, such as flax (Linum e.g. usitatissimum), cotton (Gossypium e.g. hirsutum), pine (Pinus sp.), oak (Quercus sp.), eucalyptus (Eucalyptus sp.), and the like and ornamental plants such as turfgrass (Lolium, e.g. rigidum), petunia (Petunia, e.g. x hybrida), hyacinth (Hyacinthus orientalis), carnation (Dianthus e.g. caryophyllus), delphinium (Delphinium, e.g. ajacis), Job's tears (Coix lacryma-jobi), snapdragon (Antirrhinum majus), poppy (Papaver, e.g. nudicaule), lilac (Syringa, e.g. vulgaris), hydrangea (Hydrangea e.g. macrophylla), roses (including Gallicas, Albas, Damasks, Damask Perpetuals, Centifolias, Chinas, Teas and Hybrid Teas) and ornamental goldenrods (e.g. Solidago spp.). Generally, isolated plant cells are treated according to the methods of the invention and then used to regenerate whole plants according to any method known in the art.

Relatively few specific plant mutations that produce desirable phenotypes have been described for plant species or cultivars. However, the methods of the instant invention may be used to identify a desirable mutation in one species, for example an experimental model plant, and the desirable mutation can then be introduced in the homologous genes of other species using the methods of the invention. Further, the methods of the invention can be used to produce “knock out” mutations by modification of specific amino acid codons to produce stop codons (e.g., a CAA codon specifying glutamine can be modified at a specific site to TAA; a AAG codon specifying lysine can be modified to TAG at a specific site; and a CGA codon for arginine can be modified to a TGA codon at a specific site). Such base pair changes will terminate the reading frame and produce a defective truncated protein, shortened at the site of the stop codon. Alternatively, frameshift additions or deletions can be directed into the genome at a specific sequence to interrupt the reading frame and produce a garbled downstream protein. Such stop or frameshift mutations can be introduced to determine the effect of knocking out the protein in either plant or animal cells. Desirable phenotypes that may be obtained in plants by known gene alterations include, for example, herbicide resistance; male- or female-sterility; salt, drought, lead, freezing and other stress tolerances; altered amino acid content; altered levels or composition of starch; and altered levels or composition of oils.

Animal or plant genotypes comprising altered levels or activity of at least one protein in the RAD52 epistasis group, the mismatch repair group or the nucleotide excision repair group are another aspect of the invention. Such animals or plants are particularly suitable for directed gene alteration according to the methods of the invention and can be maintained as a useful genetic stock. The alteration in the at least one protein in the RAD52 epistasis group, the mismatch repair group or the nucleotide excision repair group will then be maintained in the genome after introducing the desired gene alteration. Optionally, the alteration in the levels or activity of at least one protein in the RAD52 epistasis group, the mismatch repair group or the nucleotide excision repair group may, for example, be removed at the time of the gene alteration; removed subsequent to the gene alteration; or removed by conventional breeding.

A further embodiment of the invention is an assay to identify inhibitors of a protein encoded by a RAD1, RAD51, RAD52, RAD57 or PMS1 gene or suppressors of a RAD1, RAD51, RAD52, RAD57 or PMS1 gene expression. Such assay methods comprise contacting a sample with an oligonucleotide in a system known to provide for gene alteration and measuring whether the amount of gene alteration is less, more, or the same as in the absence of sample. Many suitable assay systems will be apparent to one of skill in the art, including antibiotic resistance (e.g. tetracycline, kanamycin or hygromycin), GFP and FlAsH systems disclosed herein and in International Patent Application No. PCT/US01/09761.

Another embodiment of the invention is a method to identify and optimize the genotype with respect to the ability to achieve enhanced gene alteration efficiency for an oligonucleotide that introduces a desired target gene alteration. Yet another embodiment of the invention is a kit for identifying optimum genetic background for mutagenizing a particular target. Such kit may comprise a gene altering oligonucleotide and one or more cells or cell-free extracts as described for use in the methods of the invention. In a preferred embodiment, a kit for identifying the optimum genetic background for mutagenizing a particular target comprises a collection of cell strains with mutations in each of the RAD1, RAD51, RAD52, RAD57 or PMS1 gene or combinations thereof. Cells for use in the kits of the invention include, for example, cells from any organism including bacteria, fungi, plants, and animals, including humans or other mammals. Cells for use in the kits of the invention include, for example, cultured cells of human liver, lung, colon, cervix, kidney, epithelium, COS-1 and COS-7 cells (African green monkey), CHO-K1 cells (Chinese hamster ovary), H1299 cells (human epithelial carcinoma, non-small cell lung cancer), C1271 (immortal murine mammary epithelial cells), MEF (mouse embryonic fibroblasts), HEC-1-A (human uterine carcinoma), HCT15 (human colon cancer), HCT116 (human colon carcinoma), LoVo (human colon adenocarcinoma), and HeLa (human cervical carcinoma) cancer cells as well as PG12 cells (rat pheochromocytoma) and ES cells (human embryonic stem cells). In other embodiments the cells for use in the kits of the invention can be yeast or other fungal cells, or cells from a plant, including, for example, maize, rice, wheat, barley, soybean, cotton, and potato. Other example plants include those described elsewhere herein.

Another embodiment of the invention is a kit for mutagenesis comprising a cell or a cell-free extract depleted for at least one protein or protein activity, the protein encoded by a RAD1, RAD51, RAD52, RAD57 or PMS1 gene. Depletion of the at least one protein or protein activity can be achieved by any method known in the art or described herein including, for example, purifying the cell-free extract from an organism with a mutation in at least one gene or purifying the cell-free extract from a wild-type organism and subsequently depleting the protein or the activity of the at least one protein. The cell or cell-free extract for the kit of the invention may be derived from any organism. In a preferred embodiment the cell or cell-free extract is or is from a eukaryotic cell or tissue. In a more preferred embodiment the cell or cell-free extract is or is from a yeast cell.

### BRIEF DESCRIPTION OF THE DRAWINGS

FIG. 1. Genetic readout system for correction of a point mutation in plasmid pK^{S}m4021. A mutant kanamycin gene harbored in plasmid pK^{S}m4021 is the target for correction by oligonucleotides. The mutant G is converted to a C by the action of the oligonucleotide. Corrected plasmids confer resistance to kanamycin in E.coli (DH10B) after electroporation leading to the genetic readout and colony counts. The sequence of chimeric, RNA-DNA double-hairpin oligonucleotide KanGG is shown (SEQ ID NO: 1).

FIG. 2. Hygromycin-eGFP target plasmids. Diagram of plasmid pAURHYG(×)eGFP. Plasmid pAURHYG(rep)eGFP contains a base substitution mutation introducing a G at nucleotide 137, at codon 46, of the Hygromycin B coding sequence (cds). Plasmid pAURHYG(ins)eGFP contains a single base insertion mutation between nucleotides 136 and 137, at codon 46, of the Hygromycin B coding sequence (cds) which is transcribed from the constitutive ADH1 promoter. Plasmid pAURHYG(Δ)eGFP contains a deletion mutation removing a single nucleotide at codon 46, of the Hygromycin B coding sequence (cds). The target sequence presented below indicates the deletion of an A and the substitution of a C for a T directed by the oligonucleotides to re-establish the resistant phenotype. The target sequence presented below the diagram indicates the amino acid conservative replacement of G with C, restoring gene function. The sequences of the normal hygromycin resistance allele (SEQ ID NO: 2) and the desired allele after gene alteration (SEQ ID NO: 3) are shown next to the mutant alleles present in pAURHYG(rep)eGFP (SEQ ID NO: 4), pAURHYG(ins)eGFP (SEQ ID NO: 5) and pAURHYG(Δ)eGFP (SEQ ID NO: 6). The position of the deletion in the pAURHYG(Δ)eGFP allele is indicated with a the symbol Δ.

FIG. 3. Oligonucleotides for correction of hygromycin resistance gene. The sequence of the oligonucleotides used in experiments to assay correction of a hygromycin resistance gene are shown. DNA residues are shown in capital letters, RNA residues are shown in lowercase and nucleotides with a phosphorothioate backbone are capitalized and underlined. In FIG. 3, the sequence of HygE3T/25 corresponds to SEQ ID NO: 7, the sequence of HygE3T/74 corresponds to SEQ ID NO: 8, the sequence of HygE3T/74a corresponds to SEQ ID NO: 9, the sequence of HygGG/Rev corresponds to SEQ ID NO: 10 and the sequence of Kan70T corresponds to SEQ ID NO: 11.

FIG. 4. pAURNeo(−)FlAsH plasmid. This figure describes the plasmid structure, target sequence, oligonucleotides, and the basis for detection of the gene alteration event by fluorescence. In FIG. 9, the sequence of the Neo/kan target mutant corresponds to SEQ ID NO: 12 and SEQ ID NO: 13, the converted sequence corresponds to SEQ ID NO: 14 and SEQ ID NO: 15 and the FlAsH peptide sequence corresponds to SEQ ID NO: 16.

FIG. 5. Fluorescent microscopy figures of targeting in the FlAsH system. This figure shows confocal microscopy of yeast strains before and after transfection by DNA/RNA CO kanGGrv. Converted yeast cells are indicated by bright green fluorescence. (A) Upper left: wild type (mat-alpha), nontargeted. Upper right: ΔRAD52, nontargeted. (C) Lower left: wild type (mat-alpha), targeted. (D) Lower right: ΔRAD52, targeted.

FIG. 6. pYESHyg(×)eGFP plasmid. This plasmid is a construct similar to the pAURHyg(×)eGFP construct shown in FIG. 7, except the promoter is the inducible GAL1 promoter. This promoter is inducible with galactose, leaky in the presence of raffinose, and repressed in the presence of dextrose.

### EXAMPLE 1

DNA Repair Genes Influence the Ability to Direct Gene Alteration in vitro

In this example, we use single-stranded oligonucleotides with modified backbones or double-hairpin oligonucleotides with chimeric, RNA-DNA backbones to measure gene alteration of episomal target sequences in cell-free extracts from cells with increased or decreased expression of DNA repair genes. These target sequences encode, for example, a kanamycin resistance gene (pKan^{S}m402l), a tetracycline resistance gene, and a fusion between a hygromycin resistance gene and eGFP. In each case, the target gene is non-functional due to at least one point mutation in the coding region.

Preparation and use of cell-free extracts for gene alteration experiments. We grow yeast cells into log phase (OD₆₀₀=0.5-0.8) in 2L YPD medium at 30° C. We then centrifuge the cultures at 5000xg, resuspend the pellets in a 10% sucrose, 50 mM Tris, 1 mM EDTA lysis solution and freeze them on dry ice. After thawing, we add KCI, spermidine and lyticase to final concentrations of 0.25 mM, 5 mM and 0.1 mg/ml, respectively. We incubate the suspension on ice for 60 minutes, add PMSF and Triton X100 to final concentrations of 0.1 mM and 0.1% and continue to incubate on ice for 20 minutes. We centrifuge the lysate at 3000×g for 10 minutes to remove larger debris. We then remove the supernatant and clarify it by centrifuging at 30000×g for 15 minutes. We then add glycerol to the clarified extract to a concentration of 10% (v/v) and freeze aliquots at −80° C. We determine the protein concentration of the extract by the Bradford assay.

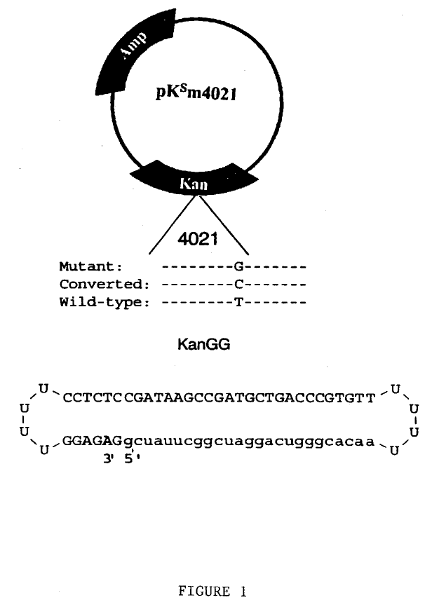

To assay gene alteration activity, we use 50 μl reaction mixtures comprising 10-30 μg protein of cell-free extract from either a wild-type yeast strain or a yeast strain having a mutation in a gene from the RAD52 epistasis group, the mismatch repair group or the nucleotide excision repair group; about 1.5 μg chimeric double-hairpin oligonucleotide (KanGG, see FIG. 1) or 0.55 μg single-stranded molecule (3S/25G or 6S/25G, 25-mer oligonucleotides directing the same alteration as KanGG and having 3 or 6 phosphorothioate linkages at each end, respectively); and about 1 μg of plasmid DNA (see FIG. 1) in a reaction buffer comprising 20 mM Tris pH 7.4, 15 mM MgCl₂, 0.4 mM DTT, and 1.0 mM ATP. We initiate the reactions by adding cell-free extract and incubating at 30° C. for 45 min. We stop the reaction by placing the tubes on ice and then immediately deproteinize them with two phenol/chloroform (1:1) extractions. We then ethanol precipitate the samples and pellet the nucleic acid at 15,000 r.p.m. at 4° C. for 30 min; wash the pellet with 70% ethanol; resuspend the nucleic acid in 50 μl H₂O; and store it at −20° C.

We measure the effect of oligonucleotide concentration on gene alteration in cell-free extract as follows. We use about 1 μg of plasmid pK^{S}m4021 and varying amounts of oligonucleotide in a 100 μl reaction mixtures comprising 20 mM Tris pH 7.6; 15 mM MgCl₂; 1 mM DTT; 0.2 mM spermidine; 2.5 mM ATP; 0.1 mM each CTP, GTP, UTP; 0.01 mM each dATP, dCTP, dGTP and dTTP; 0.1 mM NAD; and 10 μg/ml BSA. We start the reactions by adding 10-80 μg of cell-free extract and incubate the reactions at 30° C. for 30 min. We stop the reactions on ice and isolate the plasmid DNA with two phenol and one chloroform extraction followed by ethanol precipitation on dry ice for 1 hr and centrifugation at 4° for 30 min. We then wash the pellet with 70% ethanol, resuspend in μl H₂O and store at −20° C.

Quantification of gene alteration. We then electroporate 5 μl of plasmid from the resuspension (˜100 ng) into 20 μl of DH10B cells in a Cell-Porator apparatus with settings of 400 V, 300 μF, 4 kΩ (Life Technologies). After electroporation, we transfer cells to a 14 ml Falcon snap-cap tube with 1 or 2 ml SOC and shake at 37° C. for 1 h. To enhance the final kanamycin resistant colony counts, we amplify plasmids with altered sequence by adding kanamycin (50 μg/mi) or 3 ml SOC with 10 μg/ml kanamycin and shake the cell suspension for 2 or 3 h more at 37° C. We then directly plate 100 μl aliquots of undiluted cultures on LB agar plates with 50 mg/ml kanamycin and 100 μl aliquots of a 10⁴ dilution on LB agar plates with 100 mg/ml ampicillin. Alternatively, we first centrifuge the cells at 3750×g and resuspend the pellet in 500 μl SOC. We add 200 μl of the resuspension (undiluted) to kanamycin (50 μg/ml) agar plates and 200 μl of a 10⁵ dilution to ampicillin (100 μg/ml) plates. After overnight 37° C. incubation, we count bacterial colonies using an Accucount 1000 (Biologics). We measure gene alteration efficiency as the ratio of the kanamycin resistant colonies to the ampicillin resistant colonies corrected for the dilution.

Alternatively, we use the following procedure. We transform 5 μl of resuspended reaction mixtures (total volume 50 μl) into 20 μl aliquots of electro-competent DH10B bacteria using a Cell-Porator apparatus (Life Technologies). We allow the mixtures to recover in 1 ml SOC at 37° C. for 1 hour at which time we add 50 μg/ml kanamycin or 12 μg/ml tetracycline (for kanamycin or tetracycline plasmids, respectively) and incubate for an additional 3 hours. Prior to plating, we pellet the bacteria and resuspend in 200 μl of SOC. We plate 100 μl aliquots on kanamycin or tetracycline agar plates and 100 μl of a 10⁻⁴ dilution of the cultures on agar plates containing 100 μg/ml of ampicillin. We determine colony counts using an Accu-count 1000 plate reader (Biologics).

For both plating procedures we generally plate in duplicate or triplicate. Each plate contains 200-500 ampicillin resistant colonies or 0-500 tetracycline or kanamycin resistant colonies. We then select resistant colonies for plasmid isolation and DNA sequencing using an ABI Prism kit on an ABI 310 capillary sequencer (PE Biosystems).

Gene alteration in cell-free extracts from yeast. We use the kanamycin plasmid assay system to test cell-free extracts from the yeast strain LSY678. As shown in Table 1, we observe that the reaction depends on all reaction components. We also generally observe that increasing the amount of oligonucleotide or the amount of extract in the reaction increases the relative correction efficiency. We then analyze the efficiency of gene alteration in yeast strains deficient for at least one protein from the RAD52 epistasis group, the mismatch repair group or the nucleotide excision repair group. We find that extracts produced from a yeast strain lacking MSH2 (LSY814) show a significant reduction in repair activity similar to the lower gene repair that we see in mammalian cells deficient in Msh2p (Table 2). We observe that cell-free extracts from strains lacking RAD57 or RAD59 show little change in gene alteration activity and that cell free extracts from strains lacking RAD23 or RAD54 show a slight increase in gene alteration activity relative to a strain with functional copies of these genes. However, we observe elevated gene alteration frequencies using cell-free extracts from strains deficient in RAD51 or RAD52. In particular, we observe that the ΔRAD52 (LSY386) strain exhibits about 5-fold to about 25-fold higher repair frequency. In all samples, the range of ampicillin resistance colonies is 500-600 per plate with kanamycin colonies between 10 and 300.

Gene repair depends on the dose of repair proteins. We examine the activity of an extract lacking RAD52 in more detail. First, we observe that repair of pK^{S}m4021 depends on the addition of all three components: plasmid, oligonucleotide and extract (Table 3). We also observe that the repair is dose-dependent and proportional to the amount of LSY386 (ΔRAD52) extract present in a reaction where two extracts are mixed (Table 3). We confirm that RAD52 is present in these extracts by western blot analyses. We observe a similar effect on repair in cell-free extract when a strain lacking RAD52 is mixed with a strain lacking RAD23 (YEL037C) instead of LSY678.

Finally, we analyze gene alteration efficiency of cell-free extracts from LSY386 or LSY678 containing a plasmid expressing RAD52. We observe that the expression of RAD52 reduces the level of gene alteration activity in extracts made from either LSY386 or LSY678. In LSY386, the level of repair drops to near wild-type levels while the level in LSY678 is reduced to 4-fold below wild-type levels (Table 3). We perform western blot analysis on these strains and the level of RAD52 protein expression in these strains is approximately equal. These results indicate that expression of the RAD52 gene suppresses oligonucleotide-directed gene alteration. We also analyze the DNA sequence of the target plasmid from three colonies and observe that the targeted base is precisely changed even in samples in which the extract came from ΔRAD51 or ΔRAD23. Hence, target specificity is maintained despite the mutations and the differences in gene alteration frequency.

Tables are attached hereto.

TABLE 1
Gene repair using Saccaromyces cerevisae extracts

| | | | Relative |
|---|---|---|---|
| | Chimeric | | Frequency |
| Plasmid (1 μg) | Oligonucleotide (μg) | Extract (μg) | kan^{r}/amp^{r}(× 10⁻⁵) |
| pK^{S}m4021 | 1 (Kan GG) | — | 0.002 |
| pK^{S}m4021 | — | 20 | 0.0 |
| — | 1 (Kan GG) | 20 | 0.0 |
| — | — | — | 0.0 |
| pK^{S}m4021 | 1 (Kan GG) | 1 | 0.32 |
| pK^{S}m4021 | 1 (Kan GG) | 10 | 3.66 |
| pK^{S}m4021 | 1 (Kan GG) | 20 | 7.601 |
| pK^{S}m4021 | 0.5 (Kan GG) | 10 | 1.89 |
| pK^{S}m4021 | 1.0 (Kan GG) | 10 | 2.78 |
| pK^{S}m4021 | 2.0 (Kan GG) | 10 | 4.005 |
| pK^{S}m4021 | 1 (Kan CG) | — | 0.0 |
| pK^{S}m4021 | 1 (Kan CG) | 20 | 0.003 |

Chimeric oligonucleotides at varying levels are incubated with plasmid pK^{S}m4021 and the indicated amounts of cell-free extracts from Saccharomyces cerevisae (LSY678) for 45 minutes at 30° C. We isolate, purify and electroporate the plasmids into E. coli (DH10B) and quantify resistant colonies using an automatic plate reader. Relative frequency is presented as kanamycin resistant colonies divided by ampicillin resistant colonies (×10⁻⁵). Oligonucleotide KanCG has the same sequence as KanGG except there is no mismatch and KanCGt does not correct the mutation. Each data point is presented as the average of 5 independent experiments.

TABLE 2
Gene repair using mutant strains of Saccaromyces cerevisae

| | | | Relative |
|---|---|---|---|
| | | Source of | Correction |
| Plasmid | Oligonucleotide | Extract | Efficiency |
| pK^{S}m4021 | KanGG | — | 0.0 |
| pK^{S}m4021 | — | LSY678 | 0.002 |
| pK^{S}m4021 | KanGG | LSY678 (Δmatα) | 1.17 |
| pK^{S}m4021 | KanGG | LSY814 (ΔMSH2) | 0.79 |
| pK^{S}m4021 | KanGG | LSY402 (ΔRAD51) | 5.15 |
| pK^{S}m4021 | KanGG | LSY386 (ΔRAD52) | 25.7 |
| pK^{S}m4021 | KanGG | XS827-18C (ΔRAD54) | 1.36 |
| pK^{S}m4021 | KanGG | YDR076W (ΔRAD55) | 1.27 |
| pK^{S}m4021 | KanGG | LSY407 (ΔRAD57) | 2.13 |
| pK^{S}m4021 | KanGG | LSY837 (ΔRAD59) | 0.35 |
| pK^{S}m4021 | KanGG | YELO37C (ΔRAD23) | 1.04 |

Reaction mixtures (20 μl ) containing 1 μg plasmid pK^{S}m4021 and 1 μg oligonucleotide KanGG are mixed with 10 μg of a cell-free extract from the indicated yeast strains. After a 45 minute incubation at 30° C., we isolate the plasmid DNA and electroporate into E. coli (DH10B). We count kanamycin resistant colonies on agar plates containing 50 μg/ml kanamycin. Plasmids from duplicate reaction mixtures are also electroporated into E. coli (DH10B) and plated on ampicillin containing plates. We determine relative activity by dividing Kan^{r} by Amp^{r} colony numbers. These numbers reflect an average of five reactions.

TABLE 3
Extracts from L5Y386(ΔRAD52) exhibit higher levels of gene repair.

| | | | | Relative |
|---|---|---|---|---|
| | | | | Correc- |
| | Oligonu- | Source of | Source of | tion Ef- |
| Plasmid | cleotide | First Extract | Second Extract | ficiency |
| pK^{S}m4021 | — | — | — | 0.0 |
| — | KanGG | — | — | 0.0 |
| pK^{S}m4021 | KanGG | — | — | 0.003 |
| pK^{S}m4021 | KanGG | LSY678(Δmatα) | — | 1.08 |
| pK^{S}m4021 | KanGG | LSY386(ΔRAD52) | — | 26.7 |
| pK^{S}m4021 | KanGG | LSY386(2 μg) | LSY678(8 μg) | 2.91 |
| pK^{S}m4021 | KanGG | LSY386(4 μg) | LSY678(6 μg) | 5.45 |
| pK^{S}m4021 | KanGG | LSY386(6 μg) | LSY678(4 μg) | 10.47 |
| pK^{S}m4021 | KanGG | LSY386(8 μg) | LSY678(2 μg) | 14.36 |
| pK^{S}m4021 | KanGG | LSY386(2 μg) | YELO37C(8 μg) | 1.85 |
| pK^{S}m4021 | kanGG | LSY386(4 μg) | YELO37C(6 μg) | 3.71 |
| pK^{S}m4021 | KanGG | LSY386(6 μg) | YELO37C(4 μg) | 9.22 |
| pK^{S}m4021 | KanGG | LSY386(8 μg) | YELO37C(2 μg) | 16.95 |
| pK^{S}m4021 | KanGG | LSY386 | — | 19.9 |
| pK^{S}m4021 | KanGG | LSY386 · p52 | — | 2.31 |
| pK^{S}m4021 | KanGG | LSY678 | — | 1.63 |
| pK^{S}m4021 | KanGG | LSY678 · p52 | — | 0.41 |

Reaction mixtures and processing for colonies are as described in the legend to Table 1 with the following exceptions. We use cell-free extracts from yeast strains containing mutations as follows: LSY678 (Δmatα), LSY386 (ΔRAD52), and YELO37C (ΔRAD23). We use either 10 μg of extract or the amounts indicated. The reactions identified as LSY386·p52 contain a cell-free extract from a ΔRAD52 strain (LSY386) harboring a plasmid which expresses RAD52 protein. The reactions identified as LSY678·p52 contain a cell-free extract from a Δmatα strain (LSY678) harboring a plasmid which expresses RAD52 protein.

### EXAMPLE 2

DNA Repair Genes Influence the Ability to Direct Gene Alteration in vivo

In this example, we use single-stranded oligonucleotides with modified backbones or double-hairpin oligonucleotides with chimeric, RNA-DNA backbones to measure gene alteration of target sequences in cells with increased or decreased expression of DNA repair genes. These target sequences encode, for example, a fusion between a hygromycin resistance gene and eGFP which is non-functional due to at least one point mutation in the coding region. Modifications to the oligonucleotides and construction of target vectors are disclosed in the copending International Patent Application PCT/US01/09761 of Kmiec et al. entitled “Targeted Chromosomal Genomic Alterations with Modified Single Stranded Oligonucleotides”, filed Mar. 27, 2001, the disclosure of which is hereby incorporated by reference.

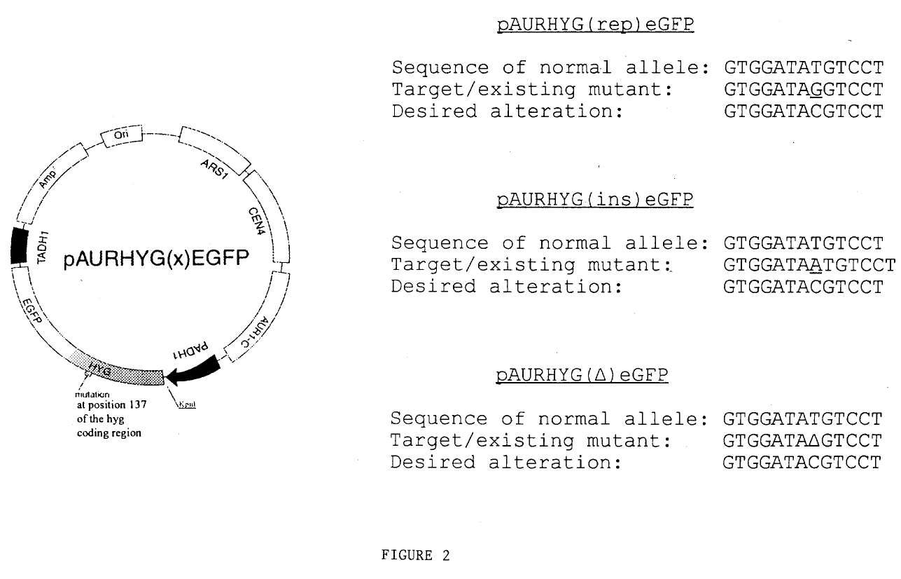

Plasmids and in vivo assay system. We employ a yeast system using the plasmids pAURHYG(rep)eGFP, which contains a point mutation in the hygromycin resistance gene, pAURHYG(ins)eGFP, which contains a single-base insertion in the hygromycin resistance gene and pAURHYG(Δ)eGFP which has a single base deletion (shown in FIG. 2). We also use the same plasmid containing a functional copy of the hygromycin-eGFP fusion gene, designated pAURHYG(wt)eGFP, as a control. These plasmids also contain an aureobasidinA resistance gene. In pAURHYG(rep)eGFP, hygromycin resistance gene function and green fluorescence from the eGFP protein are restored when a G at position 137, in codon 46 of the hygromycin B coding sequence, is converted to a C thus removing a premature stop codon in the hygromycin resistance gene coding region. In pAURHYG(ins)eGFP, hygromycin resistance gene function and green fluorescence from the eGFP protein are restored when an A inserted between nucleotide positions 136 and 137, in codon 46 of the hygromycin B coding sequence, is deleted and a C is substituted for the T at position 137, thus correcting a frameshift mutation and restoring the reading frame of the hygromycin-eGFP fusion gene. In pAURHYG(Δ)eGFP, hygromycin resistance gene function and green fluorescence from eGFP are restored when a C is inserted at the site of the single nucleotide deletion.

We synthesize the set of three yeast expression constructs pAURHYG(rep)eGFP, pAURHYG(Δ)eGFP, pAURHYG(ins)eGFP, that contain a point mutation at nucleotide 137 of the hygromycin-B coding sequence as follows: (rep) indicates a T137→G replacement, (Δ) represents a deletion of G137 and (ins) represents an A insertion between nucleotides 136 and 137. We construct this set of plasmids by excising the respective expression cassettes by restriction digest from pHyg(×)eGFP and ligation into pAUR123 (Panvera, Calif.). We digest 10 μg pAUR123 vector DNA as well as 10 μg of each pHyg(×)EGFP construct with Kpnl and Sall (NEB). We gel purify each of the DNA fragments and prepare them for enzymatic ligation. We ligate each mutated insert into pHygeGFP vector at a 3:1 molar ratio using T4 DNA ligase (Roche). We screen clones by restriction digest, confirm by Sanger dideoxy chain termination sequencing and purify plasmid DNA using a Qiagen maxiprep kit.

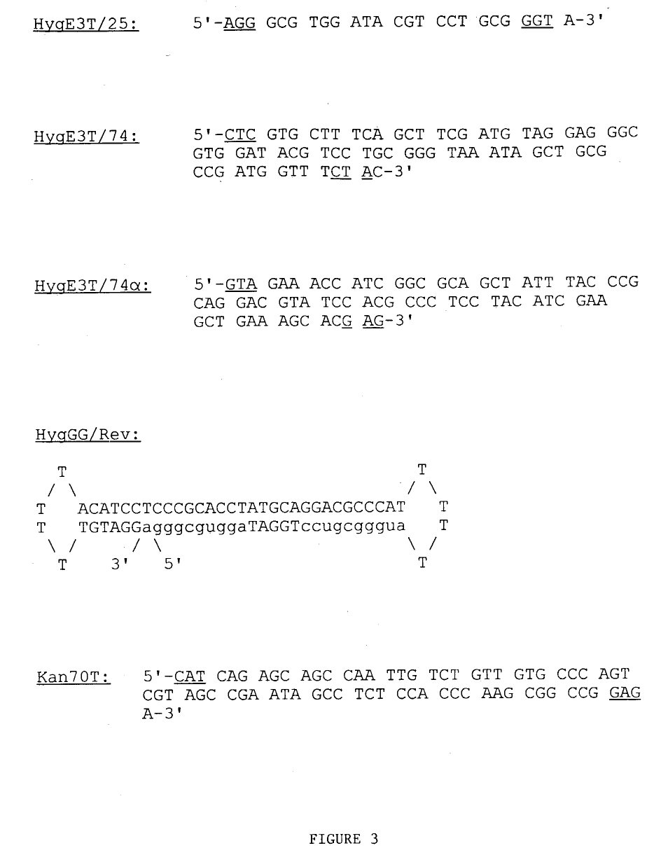

We use this system to assay the ability of modified oligonucleotides (shown in FIG. 3) to support gene alteration in a variety of host cell backgrounds including wild-type, mutants and cells expressing additional gene(s). We also use this system with chimeric RNA-DNA double-hairpin oligonucleotides. These oligonucleotides direct correction of the mutation in pAURHYG(rep)eGFP as well as the mutation in pAURHYG(ins)eGFP or pAURHYG(Δ)eGFP. The first of these oligonucleotides, HygE3T/74, is a 74-base oligonucleotide with the sequence directing gene alteration centrally positioned. The second oligonucleotide, designated HygE3T/74NT, is the reverse complement of HygE3T/74. The third oligonucleotide, designated Kan70T, is a non-specific, control oligonucleotide which is not complementary to the target sequence. Alternatively, an oligonucleotide of identical sequence but lacking a mismatch to the target or a completely phosphorothioate-modified oligonucleotide or a completely 2-O-methylated modified oligonucleotide may be used as a control.

Oligonucleotide synthesis and cells. We synthesize and purify the oligonucleotides using available phosphoramidites on controlled pore glass supports. After deprotection and detachment from the solid support, each oligonucleotide is gel-purified using, for example, procedures such as those described in Gamper et al., Biochem. 39, 5808-5816 (2000). We determine the concentration of the oligonucleotides spectrophotometrically (33 or 40 μg/ml per A₂₆₀ unit of single-stranded or hairpin oligomer, respectively). Plasmids used for assay are maintained stably at low copy number under aureobasidin selection in yeast (Saccharomyces cerevisiae) strain LSY678 MAT α which optionally may contain additional gene mutations or may be engineered to express additional protein(s).

Plasmids and oligonucleotides are introduced into yeast cells by electroporation as follows: to prepare electrocompetent yeast cells, we inoculate 10 ml of YPD media from a single colony and grow the cultures overnight with shaking at 300 rpm at 30° C. We then add 30 ml of fresh YPD media to the overnight cultures and continue shaking at 30° C. until the OD₆₀₀ was between 0.5 and 1.0 (3-5 hours). We then wash the cells by centrifuging at 4° C. at 3000 rpm for 5 minutes and twice resuspending the cells in 25 ml ice-cold distilled water. We then centrifuge at 4° C. at 3000 rpm for 5 minutes and resuspend in 1 ml ice-cold 1M sorbitol and then finally centrifuge the cells at 4° C. at 5000 rpm for 5 minutes and resuspend the cells in 120 μl 1M sorbitol. To transform electrocompetent cells with plasmids or oligonucleotides, we mix 40 μl of cells with 5 μg of nucleic acid, unless otherwise stated, and incubate on ice for 5 minutes. We then transfer the mixture to a 0.2 cm electroporation cuvette and electroporate with a BIO-RAD Gene Pulser apparatus set at 1.5 kV, 25 μF, 200 Ω for one five-second pulse. We then immediately resuspend the cells in 1 ml YPD supplemented with 1M sorbitol and incubate the cultures at 30° C. with shaking at 300 rpm for 6 hours. We then spread 200 μl of this culture on selective plates containing 300 μg/ml hygromycin and spread 200 μl of a 10⁵ dilution of this culture on selective plates containing 500 ng/ml aureobasidinA and/or hygromycin and incubate at 30° C. for 3 days to allow individual yeast colonies to grow. We then count the colonies on the plates and calculate the gene alteration efficiency by determining the number of hygromycin resistance colonies per 10⁵ aureobasidinA resistant colonies.

Gene alteration to repair different mutations in wild-type Saccharomyces cerevisiae. We test the ability of oligonucleotides shown in FIG. 3 to alter all three target plasmids in vivo using wild-type yeast strain LSY678 MAT α. These target plasmids contain a point mutation (pAURHYG(rep)eGFP), a deletion mutation (pAURHYG(Δ)eGFP) or an insertion mutation (pAURHYG(ins)eGFP). We also test oligonucleotides targeting opposite strands of the target DNA to identify any strand-specific effects and we test the oligonucleotide at a range of concentration to determine the optimum concentration for gene repair.

As shown in Table 4, we observe that oligonucleotides targeting either strand direct correction of all three types of mutations. The data indicate that the point mutation in pAURHYG(rep)eGFP is corrected more efficiently than the insertion mutation in pAURHYG(ins)eGFP, which in turn is corrected more efficiently than the deletion mutation in pAURHYG(Δ)eGFP. In addition, with all three assay plasmids we observe that the optimal oligonucleotide concentration for gene alteration in this system is 5 μg. We note, however, that the oligonucleotides are capable of effecting repair over a wide range of concentrations. Finally, we observe that the oligonucleotide with sequence complementary to the sense strand of the target DNA, HygE3T/74NT, repairs all three types of target mutations more effectively than the complementary oligonucleotide, HygE3T/74. The fold difference in repair efficiency using HygE3T/74NT relative to using HygE3T/74 is indicated in the final column of Table 4.

Gene alteration in strains with mutation(s) in gene(s) of the RAD52 epistasis group. We test the ability of oligonucleotides shown in FIG. 3 to alter a nucleic acid sequence in vivo using yeast strains with additional mutation(s) in gene(s) of the RAD52 epistasis group. In these experiment we used derivatives of LSY678 MAT α with a mutation in one or more of the genes of the RAD52 epistasis group and containing the target plasmid pAURHYG(rep)eGFP, pAURHYG(ins)eGFP or pAUR HYG(Δ)eGFP. We electroporated these cells with 5 μg of HygE3T/74 and plated on hygromycin and aureobasidinA to obtain the efficiency of gene alteration. The results of these experiments for plasmid pAURHYG(rep)eGFP, pAURHYG(ins)eGFP and pAUR HYG(Δ)eGFP are shown in Table 5, Table 6 and Table 7, respectively.

These data indicate that the efficiency of gene alteration is reduced or unchanged in a yeast strain with a mutation in RAD51, RAD52, RAD54, RAD55, RAD59, RAD50, MRE11 or XRS2. The efficiency of gene alteration that we observe in these experiments in strains with mutations in either RAD57 or a double mutant in RAD51/52 is reduced when using pAURHYG(ins)eGFP or pAUR HYG(Δ)eGFP as the target plasmid, but, surprisingly, we observe an increase in the efficiency of gene alteration in these strains when using pAURHYG(rep)eGFP as the target. We observe that gene alteration using pAURHYG(rep)eGFP as the target is reduced in a yeast strain with a mutation in RAD54 or RAD55. We also perform control experiments with LSY678 yeast cells containing the plasmid pAURHYG(wt)eGFP. With this strain we observe that even without added oligonucleotides, there are too many hygromycin resistant colonies to count. We test yeast strains with mutations in both single genes in the RAD52 epistasis group as well as yeast strains with mutations in two or more of the genes. We test the ability of these yeast strains to correct all of the pAURHYG(×)eGFP mutations.

Gene alteration in strains with mutation(s) in mismatch repair gene(s). We test the ability of oligonucleotides shown in FIG. 3 to alter a nucleic acid sequence in vivo using yeast strains with additional mutation(s) in mismatch repair gene(s) containing the plasmid pAURHYG(×)GFP. We electroporate these cells with 5 μg of HygE3T/74 and plated on hygromycin and aureobasidinA to obtain the efficiency of gene alteration. For example, the results of these experiments for plasmid pAURHYG(rep)eGFP, pAURHYG(ins)eGFP and pAUR HYG(Δ)eGFP are shown in Table 5, Table 6 and Table 7, respectively.

These data indicate that gene alteration occurs at a reduced efficiency in strains with mutations in MSH2, MSH3 or MSH6 and at an increased efficiency in strains with a mutation in PMS1. We observe the same general effects, although at different relative efficiencies, in experiments using either plasmid pAURHYG(rep)eGFP, plasmid pAURHYG(ins)eGFP or pAUR HYG(Δ)eGFP as the target. In control experiments with LSY678 yeast cells containing the plasmid pAURHYG(wt)eGFP, we again observe that, even without added oligonucleotides, there are too many hygromycin resistant colonies to count. We test yeast strains with mutations in both single mismatch repair genes as well as yeast strains with mutations in two or more of the genes. We test the ability of these yeast strains to correct all of the pAURHYG(×)eGFP mutations.

Gene alteration in strains with mutation(s) in nucleotide excision repair gene(s). We test the ability of oligonucleotides shown in FIG. 3 to alter a nucleic acid sequence in vivo using yeast strains with additional mutation(s) in nucleotide excision repair gene(s) containing the plasmid pAURHYG(×)eGFP. We electroporated these cells with 5 μg of HygE3T/74 and plated on hygromycin and aureobasidinA to obtain the efficiency of gene alteration. For example, the results of these experiments for plasmid pAURHYG(rep)eGFP, pAURHYG(ins)eGFP and pAUR HYG(Δ)eGFP are shown in Table 5, Table 6 and Table 7, respectively.

These data indicate that gene alteration occurs at a reduced efficiency in strains with mutations in RAD10, RAD2, or RAD23. The efficiency of gene alteration observed in these experiments in a strain with a mutation in RAD1 is reduced when using either pAURHYG(ins)eGFP or pAUR HYG(Δ)eGFP as the target plasmid, but increased when using pAURHYG(rep)eGFP as the target. We observe that gene alteration is reduced in a yeast strain with a mutation in EXO1 using pAURHYG(rep)eGFP or pAURHYG(ins)eGFP as the target. We also perform control experiments with LSY678 yeast cells containing the plasmid pAURHYG(wt)eGFP which yield too many hygromycin resistant colonies to count. We test yeast strains with mutations in both single nucleotide excision repair genes as well as yeast strains with mutations in two or more of the genes. We test the ability of these yeast strains to correct all of the pAURHYG(×)eGFP mutations.

We also use additional oligonucleotides to assay the ability of individual oligonucleotides to correct multiple mutations in the pAURHYG(×)eGFP plasmid contained in yeast strains containing mutations in genes of the RAD52 epistasis group, genes involved in mismatch repair and/or genes involved in nucleotide excision repair. These include, for example, one that alters two basepairs that are 3 nucleotides apart is a 74-mer with the sequence 5′-CTCGTGCTTTCAGCTTCGATGTAGGAGGGCGTGGGTACGTCCTGCGGGTAAATAGCTGCGCCGATG GTTTCTAC-3′ (SEQ ID NO: 17); a 74-mer that alters two basepairs that are 15 nucleotides apart with the sequence 5′-CTCGTGCTTTCAGCTTCGATGTAGGAGGGCGTGGATACGTCCTGCGGGTAAACAGCTGCGCCGATG GTTTCTAC-3′ (SEQ ID NO: 18); and a 74-mer that alters two basepairs that are 27 nucleotides apart with the sequence 5′-CTCGTGCTTTCAGCTTCGATGTAGGAGGGCGTGGATACGTCCTGCGGGTAAATAGCTGCGCCGACG GTTTCTAC (SEQ ID NO: 19). The nucleotides in these oligonucleotides that direct alteration of the target sequence are underlined and in boldface. These oligonucleotides are modified in the same ways as the previously described oligonucleotides.

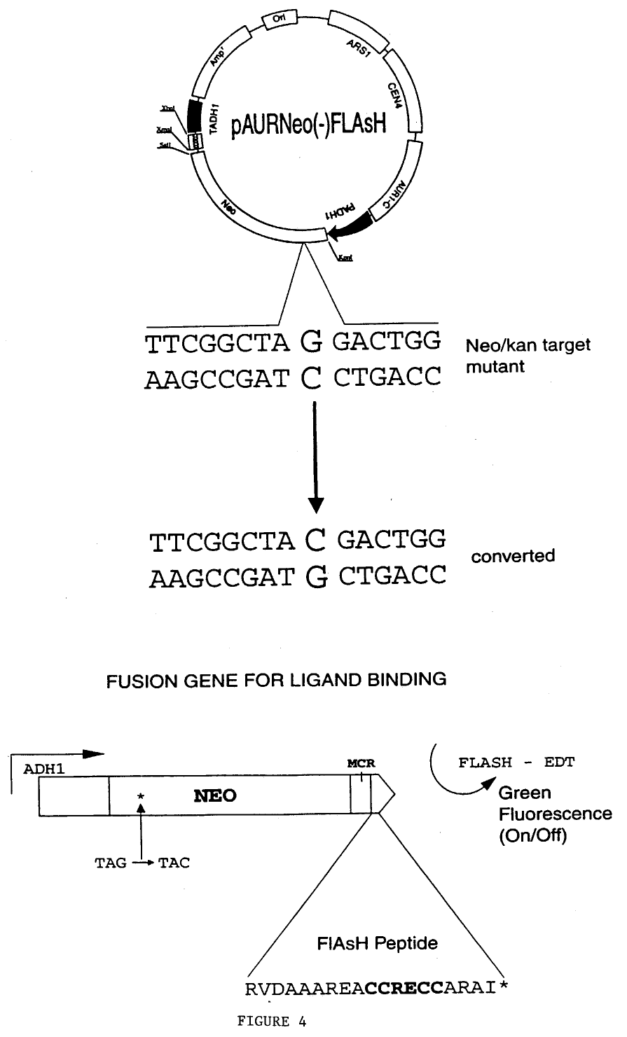

We also test the ability of oligonucleotides shown in FIG. 1 to alter a nucleic acid sequence in vivo using yeast strains with additional mutation(s) in mismatch repair gene(s) containing the plasmid pAURNeo(×)FlAsH (FIG. 4). This plasmid was constructed by inserting a synthetic expression cassette containing a neomycin phosphotransferase (kanamycin resistance) gene and an extended reading frame that encodes a receptor for the FlAsH ligand into the pAUR123 shuttle vector (Panvera Corp., Madison, Wis.). We make constructs with the same mutation as in pK^{S}m4021. The resulting construct replicates in S. cerevisiae at low copy number, confers resistance to aureobasidinA and constitutively expresses the Neo(×)FlAsH fusion product from the ADH1 promoter. By extending the reading frame of this gene to code for a unique peptide sequence capable of binding a small ligand to form a fluorescent complex, restoration of expression by correction of the stop codon can be detected in real time using confocal microscopy. Upon correction of the truncated Neo(−)FlAsH product to generate the Neo(+)FlAsH fusion product the translated fusion protein binds a ligand (FlAsH-EDT2) imparting a green fluorescence onto the cells. Additional constructs using any target gene fused to the FlAsH peptide may be made using this model system to test additional gene alteration events.

To detect the presence of the Neo(+)FlAsH fusion product in yeast cells, we prepare loading buffer by mixing FlAsH ligand into YPD containing 1M sorbitol and 20 μM Disperse 3. The ligand molecules are mixed into the YPD at 1 μM FlAsH EDT2 and 10 μM 1,2 ethanedithiol (EDT) (Sigma). We then mix 100 μl of cells with an equal volume of wash buffer comprising HBS, 1 mM sodium pyruvate, 10 μM EDT, 1 M sorbitol and 20 μM Disperse 3. We then image the cells with a Zeiss LSM510 laser scanning microscope on a Zeiss Axiovert 100 m using the 488/568 nm excitation line of an Omnichrome Ar—Kr laser with appropriate emission filters (505-550 nm bandpass for FlAsH-EDT2 binding). We simultaneously acquire laser scanning transmitted or differential interference contrast images with all fluorescent images using 488 nm excitatory. We load samples into a Lab-Tek II chambered #1.5 Coverglass system (Nalge Nunc International, Ill.) and image them using a Zeiss 63× C-Apochromet water immersion lens (NA 1.2). All samples, including positive and negative controls, are integrated under identical conditions (laser power, pinhole, PMT gap offset, etc.) for a given set of experiments.

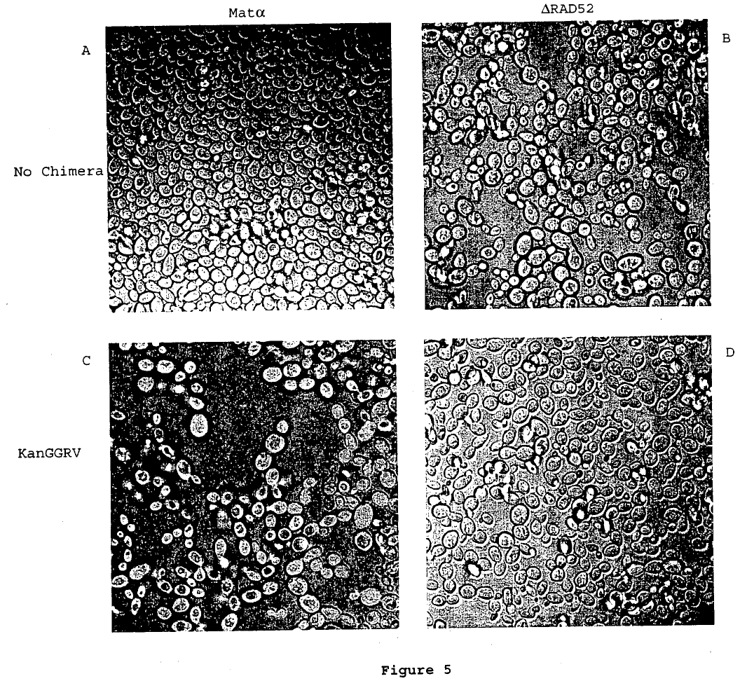

We observe correction of a mutation in the neomycin phosphotransferase gene (Neo) harbored in yeast strain LSY678 using a FlAsH-EDT2 model system. We electroporate KanGG into either LSY386 or LSY678 containing stable copies of the pAURNeo(-)FlAsH plasmid. We measure uptake of oligonucleotide using Texas Red conjugated oligonucleotide and optimize electroporation conditions so that over 80% of the surviving cells receive the oligonucleotide. In the absence of KanGG, we observe only a low level of auto-fluorescence after addition of FlAsH-EDT2 in both LSY678 (FIG. 5A) and LSY386 (FIG. 5B) by confocal microscopy. However, when we introduce KanGG into the cells, we observe many corrected cells in both LSY678 and LSY378 as seen in FIG. 5C and FIG. 5D, respectively. We see a significant increase in the number of cells exhibiting green fluorescence in the LSY378 strain lacking RAD52 (FIG. 5D) relative to the LSY678 strain (FIG. 5C). This result reflects a higher degree of gene repair in the strain lacking RAD52 gene function. Correction of pAURNeo(−)FlAsH also confers resistance to G418 selection in yeast cells. Therefore we grow representative samples exhibiting green fluorescence on agar plates containing G418. We then determine the DNA sequence of the plasmid in these cells. The sequence analysis illustrates that the targeted base is changed from a G to a C as designed in plasmids isolated after G418 selection.

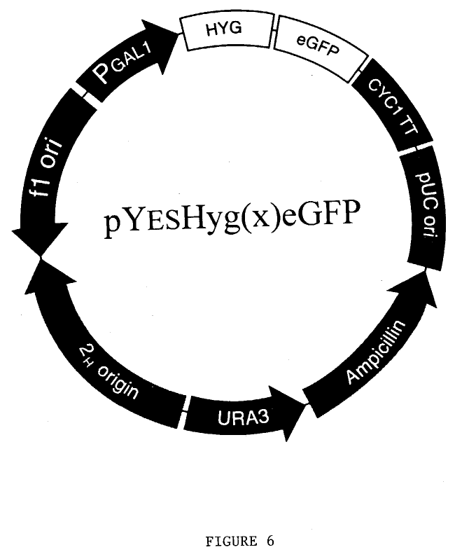

Oligonucleotides targeting the sense strand direct gene alteration more efficiently in yeast mutants. We compare the ability of single-stranded oligonucleotides to target each of the two strands of the target sequence of pAURHYG(ins)eGFP, pAURHYG(rep)eGFP or pAURHYG(Δ)eGFP present in LSY678 mutant strains with increased or decreased expression of DNA repair genes. For example, the results of an experiment performed with yeast strains having mutations in RAD1 and RAD10 are presented in Table 8. The data from this experiment indicate that an oligonucleotide, HygE3T/74NT, with sequence complementary to the sense strand (i.e. the strand of the target sequence that is identical to the mRNA) of the target sequence facilitates gene correction approximately ten-fold more efficiently than an oligonucleotide, HygE3T/74, with sequence complementary to the non-transcribed strand which serves as the template for the synthesis of RNA. However, regardless of the reduced efficiency observed in yeast strains with mutations in DNA repair genes, the oligonucleotides are clearly still able to target either strand of the target sequence. In addition, the role of transcription of the target gene is investigated using plasmids with inducible promoters such as that described in FIG. 6.

Expression of DNA repair genes. We test the effect on gene alteration efficiency of increasing expression of DNA repair genes, including genes in the RAD52 epistasis group, mismatch repair genes and nucleotide excision repair genes. We test the effect of expression of these genes both individually and in groups of two or more. We generally employ plasmids with inducible promoters, for example the plasmid described in FIG. 6, directing expression of DNA repair genes. Alternatively, we use plasmids with constitutive promoters to direct expression of DNA repair genes. We observe that increasing expression of DNA repair genes, like mutation in these genes, influences the efficiency of gene alteration in our assay system. We also test the effect of heterologous expression of DNA repair genes from other organisms, including, for example, other fungi, animals, plants and bacteria.

Influence of DNA repair genes in other cells. In addition to testing the effect of DNA repair genes in the above-described yeast assay system, we test the effect of altering the expression or the activity of DNA repair genes in other cells, including, for example, other fungi, animal, plant and bacterial cells. We use other cells with normal DNA repair genes as well as cells that have mutations in DNA repair genes, including, for example, human and bacterial cells with mutations in the homologous genes. We use cells that are transiently or stably transformed with vectors that express either native or heterologous DNA repair genes. To monitor gene alteration in these cells, we employ a reporter-gene assay system, for example, kanamycin resistance, hygromycin resistance or GFP expression. Alternatively, we assay the ability of an oligonucleotide to direct gene alteration of a target present in the genome of the target cell, for example, we monitor conversion of the sickle T (β^{S}) mutation in the β-globin gene to the normal A (β^{A}) allele or vice-versa.

Tables are attached hereto.

TABLE 4
Gene repair of different mutations in wild-type
Saccharomyces cerevisiae

| Amount of Oligonucleotide | Correcting Oligonucleotide Tested | | |
|---|---|---|---|
| (μg) | HygE3T/74 | HygE3T/74NT | Fold |

Repair of pAURHYG(rep)GFP

| | | | |
|---|---|---|---|
| 0 | 0* | 0 | 0x |
| 1.0 | 5 (0.03) | 238 (1.47) | 47.6x |
| 2.5 | 99 (0.61) | 704 (4.37) | 7.1x |
| 5.0 | 204 (1.26) | 1406 (8.73) | 6.8x |
| 7.5 | 69 (0.42) | 998 (6.20) | 14.5x |
| 10.0 | 19 (0.12) | 261 (1.62) | 13.7x |

Repair of pAURHYG(Δ)GFP

| | | | |
|---|---|---|---|
| 0 | 0 | 0 | 0x |
| 1.0 | 1 (0.01) | 1 (0.01) | 1.0x |
| 2.5 | 18 (0.11) | 68 (0.42) | 3.8x |
| 5.0 | 70 (0.43) | 308 (1.91) | 4.4x |
| 7.5 | 47 (0.29) | 276 (1.71) | 5.9x |
| 10.0 | 11 (0.07) | 137 (0.85) | 12.5x |

Repair of pAURHYG(ins)GFP

| | | | |
|---|---|---|---|
| 0 | 0 | 0 | 0x |
| 1.0 | 5 (0.03) | 45 (0.28) | 9.0x |
| 2.5 | 47 (0.29) | 387 (2.4) | 8.2x |
| 5.0 | 199 (1.24) | 623 (3.87) | 3.1x |
| 7.5 | 54 (0.34) | 398 (2.47) | 7.4x |
| 10.0 | 17 (1.10) | 271 (1.68) | 15.9x |

*Average colony count on hygromycin plates from four experiments is shown. Numbers in parentheses indicate the number of hygromycin-resistant colonies per aureobasidin-resistant colony.

TABLE 5
Gene alteration directing correction of the mutation in
pAURHYG(rep)GFP

| | | Colonies on | | |
|---|---|---|---|---|
| | Colonies on | Aureobasidin | Correction | |
| Yeast Strain | Hygromycin | (/10₅) | Efficiency | Fold |
| MAT α wild type | 1218 | 286 | 4.26 | 1x |

RAD52 Epistasis Group Mutants

| | | | | |
|---|---|---|---|---|
| RAD51 | 104 | 168 | 0.62 | 0.14x |
| RAD52 | 266 | 81 | 3.29 | 0.77x |
| RAD51/52 | 212 | 39 | 5.45 | 1.28x |
| RAD54 | 2 | 103 | 0.02 | 0x |
| RAD55 | 0 | 1230 | 0 | 0x |
| RAD57 | 984 | 57 | 17.26 | 4.05x |
| RAD59 | 1198 | 392 | 3.06 | 0.71x |
| MRE11 | 12 | 18 | 0.63 | 0.15x |
| RAD50 | 336 | 58 | 2.09 | 0.49x |
| XRS2 | 29 | 44 | 0.66 | 0.15x |

Mismatch Repair Group Mutants

| | | | | |
|---|---|---|---|---|
| MSH2 | 0 | 976 | 0 | 0x |
| MSH3 | 0 | 1035 | 0 | 0x |
| MSH6 | 1270 | 541 | 2.35 | 0.55x |
| PMS1 | 2280 | 20 | 114 | 26.76x |

Nucleotide Excision Repair Mutants

| | | | | |
|---|---|---|---|---|
| RAD1 | 1380 | 391 | 8.52 | 2.00x |
| RAD10 | 54 | 361 | 0.15 | 0.04x |
| RAD2 | 919 | 243 | 3.78 | 0.89x |
| RAD23 | 66 | 151 | 0.44 | 0.10x |
| EXO1 | 486 | 124 | 3.92 | 0.92x |

TABLE 6
Gene alteration directing correction of the muation in
pAURHYG(ins)GFP

| | Colonies on | Colonies on | Correction | |
|---|---|---|---|---|
| Yeast Strain | Hygromycin | Aureobasidin (/10₅) | Efficiency | Fold |
| MAT α wild type | 256 | 74 | 3.46 | 1x |

RAD52 Epistasis Group Mutants

| | | | | |
|---|---|---|---|---|
| RAD51 | 19 | 32 | 0.59 | 0.17x |
| RAD52 | 31 | 36 | 0.86 | 0.24x |
| RAD51/52 | 3 | 86 | 0.3 | 0.01x |
| RAD54 | 0 | 170 | 0 | 0x |
| RAD55 | 0 | 32 | 0 | 0X |
| RAD57 | 34 | 103 | 0.33 | 0.10x |
| RAD59 | 116 | 47 | 2.47 | 0.71x |
| RAD50 | 3 | 34 | 0.09 | 0.03x |
| MRE11 | 1 | 17 | 0.06 | 0.02x |
| XRS2 | 6 | 168 | 0.04 | 0.01x |

Mismatch Repair Group Mutants

| | | | | |
|---|---|---|---|---|
| MSH2 | 0 | 51 | 0 | 0x |
| MSH3 | 1 | 18 | 0.05 | 0.02x |
| MSH6 | 0 | 49 | 0 | 0x |
| PMS1 | 111 | 6 | 18.5 | 5.35x |

Nucleotide Excision Repair Mutants

| | | | | |
|---|---|---|---|---|
| RAD1 | 52 | 88 | 0.59 | 0.17x |
| RAD10 | 14 | 101 | 0.14 | 0.04x |
| RAD2 | 113 | 63 | 1.79 | 0.52x |
| RAD23 | 1 | 144 | 0.01 | 0x |
| EXO1 | 2 | 197 | 0.01 | 0x |

TABLE 7
Gene alteration directing correction of the mutation in
pAURHYG(Δ)GFP

| Yeast Strain | Fold Alteration in Correction Efficiency |
|---|---|
| MAT α wild type | 1x |

RAD52 Epistasis Group Mutants

| | |
|---|---|
| RAD51 | 0.47x |
| RAD52 | 0.05x |
| RAD51/52 | 0.13x |
| MRE11 | 1.10x |

Mismatch Repair Group Mutants

| | |
|---|---|
| MSH2 | 0x |
| MSH3 | 0.02x |
| MSH6 | 0x |

Nucleotide Excision Repair Mutants

| | |
|---|---|
| RAD1 | 0x |
| RAD10 | 0.04x |

TABLE 8
Alteration with an oligonucleotide targeting the sense strand is more
efficient

Colonies on Hygromycin

| Yeast Strain | Kan70T | HygE3T/74 | HygE3T/74NT |
|---|---|---|---|
| RAD1 | 0 | 3 | 53 (15x)* |
| RAD10 | 0 | 2 | 14 (6x)* |

*The numbers in parentheses represent the fold increase in efficiency for targeting the non-transcribed strand as compared to the other strand of a DNA duplex that encodes a protein.

Although a number of embodiments and features have been described above, it will be understood by those skilled in the art that modification and variations of the described embodiments and features may be made without departing from either the spirit of the invention or the scope of the appended claims. The publications and patents cited herein are incorporated by reference.

What is claimed is:

1. A method of enhancing oligonucleotide-mediated gene alteration efficiency comprising using a cell or a cell-free extract with reduced levels or activity of at least one protein selected from the group consisting of a homolog, ortholog or paralog of RAD1, RAD51, RAD52, RAD57 and PMS1.

2. The method of claim 1 wherein the protein is RAD1.

3. The method of claim 1 wherein the protein is RAD51.

4. The method of claim 1 wherein the protein is RAD52.

5. The method of claim 1 wherein the protein is RAD57.

6. The method of claim 1 wherein the protein is PMS1.

7. The method of claim 1 in which the gene alteration is a deletion, insertion or replacement alteration.

8. The method of claim 1 or 7 in which the gene alteration affects 1, 2 or 3 consecutive nucleotides in a target nucleic acid.

9. The method of claim 1 or 7 in which the oligonucleotide binds to the non-transcribed strand of a gene at a target site.

10. The method of claim 1 or 7 in which the oligonucleotide is a chimeric, RNA-DNA, double-hairpin oligonucleotide or a modified single-stranded oligonucleotide.

11. The method of claim 1 or 7 in which the gene alteration is in a target nucleic acid selected from the group consisting of: plasmids, cosmids, artificial chromosomes, YACs, BACs, PLACs and BiBACs.

12. The method of claim 1 or 7 in which the cell is selected from a fungal, plant, animal, mammal or human cell.

13. The method of claim 1 or 7 in which the cell-free extract is derived from a fungal, plant, animal, mammal or human cell.

14. The method of claim 1 or 7 in which the reduced levels or activity of at least one protein comprises a mutation in a gene encoding the protein.

15. The method of claim 1 or 7 in which the reduced levels or activity of at least one protein comprises addition of an inhibitor of the activity or the expression of the targeted protein or gene.

16. A kit comprising at least one cell or at least one cell-free extract wherein the cell or cell-free extract comprises reduced protein levels or activity of at least one protein selected from the group consisting of a homolog, ortholog or paralog or RAD1, RAD51, RAD52, RAD57 and PMS1.

17. A kit comprising a collection of at least two different cells wherein each cell has reduced protein levels or activity of a different protein selected from the group consisting of a homolog, ortholog or paralog or RAD1, RAD51, RAD52, RAD57 and PMS1.

18. The kit of claim 16 or 17 further comprising an oligonucleotide capable of directing gene alteration.

19. The kit of claim 16 or 17 in which the reduced protein levels or activity comprises a mutation in the gene encoding the protein or proteins.

20. The kit of claim 16 or 17 in which the reduced protein levels or activity comprises addition of an inhibitor of the activity or the expression of the targeted protein or gene.


SEQUENCE LISTING (19 sequences)

SEQ ID NO: 1 - DNA, 70 bp, Artificial Sequence
  stem_loop (1) ... (54)
  stem_loop (55) ... (70): Description of Artificial Sequence Combined DNA/RNA oligonucleotide
gcuauucggcuaggacugggcacaauuuuttgtgcccagtcgtagccgaatagcctctccuuuuggagag

SEQ ID NO: 2 - DNA, 13 bp, Escherichia coli
gtggatatgtcct

SEQ ID NO: 3 - DNA, 13 bp, Escherichia coli
gtggatacgtcct

SEQ ID NO: 4 - DNA, 13 bp, Escherichia coli
gtggataggtcct

SEQ ID NO: 5 - DNA, 14 bp, Escherichia coli
gtggataatgtcct

SEQ ID NO: 6 - DNA, 12 bp, Escherichia coli
gtggatagtcct

SEQ ID NO: 7 - DNA, 25 bp, Artificial Sequence
  misc_feature (1) ... (3)
  misc_feature (22) ... (24): Description of Artificial Sequence Oligonucleotide with phosphorothioate linkages
agggcgtggatacgtcctgcgggta

SEQ ID NO: 8 - DNA, 74 bp, Artificial Sequence
  misc_feature (1) ... (3)
  misc_feature (71) ... (73): Description of Artificial Sequence Oligonucleotide with phosphorothioate linkages
ctcgtgctttcagcttcgatgtaggagggcgtggatacgtcctgcgggtaaatagctgcgccgatggtttctac

SEQ ID NO: 9 - DNA, 74 bp, Artificial Sequence
  misc_feature (1) ... (3)
  misc_feature (72) ... (74): Description of Artificial Sequence Oligonucleotide with phosphorothioate linkages
gtagaaaccatcggcgcagctatttacccgcaggacgtatccacgccctcctacatcgaagctgaaagcacgag

SEQ ID NO: 10 - DNA, 70 bp, Artificial Sequence
  stem_loop (1) ... (54)
  stem_loop (55) ... (70): Description of Artificial Sequence Combined DNA/RNA oligonucleotide
agggcguggataggtccugcggguatttttacccgcaggacgtatccacgccctcctacatttttgtagg

SEQ ID NO: 11 - DNA, 70 bp, Artificial Sequence
  misc_feature (1) ... (3)
  misc_feature (67) ... (69): Description of Artificial Sequence Oligonucleotide with phosphorothioate linkages
catcagagcagccaattgtctgttgtgcccagtcgtagccgaatagcctctccacccaagcggccggaga

SEQ ID NO: 12 - DNA, 15 bp, Escherichia coli
ttcggctaggactgg

SEQ ID NO: 13 - DNA, 15 bp, Escherichia coli
aagccgatcctgacc

SEQ ID NO: 14 - DNA, 15 bp, Escherichia coli
ttcggctacgactgg

SEQ ID NO: 15 - DNA, 15 bp, Escherichia coli
aagccgatgctgacc

SEQ ID NO: 16 - protein, 19 aa, Artificial Sequence
  Description of Artificial Sequence FlAsH peptide sequence
RVDAAAREACCRECCARAI

SEQ ID NO: 17 - DNA, 74 bp, Artificial Sequence
  Description of Artificial Sequence Oligonucleotide
ctcgtgctttcagcttcgatgtaggagggcgtgggtacgtcctgcgggtaaatagctgcgccgatggtttctac

SEQ ID NO: 18 - DNA, 74 bp, Artificial Sequence
  Description of Artificial Sequence Oligonucleotide
ctcgtgctttcagcttcgatgtaggagggcgtggatacgtcctgcgggtaaacagctgcgccgatggtttctac

SEQ ID NO: 19 - DNA, 74 bp, Artificial Sequence
  Description of Artificial Sequence Oligonucleotide
ctcgtgctttcagcttcgatgtaggagggcgtggatacgtcctgcgggtaaatagctgcgccgacggtttctac